(19) United States
(12) Patent Application Publication
(10) Pub. No.: US 2009/0275809 A1
(43) Pub. Date: Nov. 5, 2009

(54) Portable Modular Kiosk Based Physiologic Sensor System with Display and Data Storage for Clinical and Research Applications including Cross Calculating and Cross Checked Physiologic Parameters Based Upon Combined Sensor Input

(75) Inventor: Eric W. Starr, Allison Park, PA (US)

Correspondence Address: BLYNN L. SHIDELER;THE BLK LAW GROUP, 3500 BROKKTREE ROAD, SUITE 200, WEXFORD, PA, 15090, US

(73) Assignee: STARR LIFE SCIENCES CORP., Oakmont, PA (US)

(21) Appl. No.: 12/434,599

(22) Filed: May 1, 2009

Related U.S. Application Data
(60) Provisional application No. 61/049,451, filed on May 1, 2008.

(51) Int. Cl. A61B 5/00

(52) U.S. Cl. 600/301

(57) ABSTRACT

A portable modular kiosk based physiologic sensor system for clinical and research applications configured to simultaneously utilize multiple sensors with cross checking and cross calculation of physiologic parameters.

### RELATED APPLICATIONS

The present application claims the benefit of U.S. Provisional Patent Application Ser. No. 61/049,451 entitled “Portable Modular Kiosk Based Physiologic Sensor System for Clinical and Research Applications Configured to Simultaneously Utilize Multiple Sensors with Cross Checking and Cross Calculation of Physiologic Parameters” filed May 1, 2008.

### BACKGROUND OF THE INVENTION

1. Field of the Invention

The present invention relates to a portable, modular physiologic sensor system with display and data storage for clinical and research applications, in particular to animal applications for a portable modular kiosk based physiologic sensor system for clinical and research applications configured to simultaneously utilize multiple sensors with cross checking and cross calculation of physiologic parameters.

2. Background Information

The present invention relates to monitoring of physiologic parameters of a patient or subject, in particular an animal patient or subject, such as a small mammal. The following definitions will be helpful in explaining the known background elements that are helpful for understanding the present invention. Physiologic parameters are measured with physiologic sensors that typically, but not always, contact the patient or subject. The term patient is appropriate in the medical fields for both the human medical field and animal veterinarian fields. The term subject is appropriate in the research field, and the term subject will apply to human and non-human applications.

The list of physiologic sensors is large and constantly growing. A representative list of physiologic sensors known in the art include blood pressure sensors, blood flow sensors, blood glucose sensors, blood cholesterol sensors, heart sound sensors, EMG sensors, EEG sensors, EKG sensors, EOG sensors, pulse sensors, oxygenation sensors, blood perfusion sensors, respiration monitors, temperature sensors, blood gas sensors, motion sensors, strain gauges, body position sensors, and limb motion sensors.

A “kiosk’ within this application, sometimes called an electronic kiosk, computer kiosk or interactive kiosk, houses a computer terminal that often employs custom kiosk software designed to function, hopefully flawlessly, while preventing users from accessing system functions. “Kiosk mode” is a euphemism for such a mode of software operation. Computerized kiosks may store data locally, or retrieve it from a computer network. Some common computer kiosks provide a free, informational public service while others common computer kiosks serve a commercial purpose. Touch-screens, trackballs, computer keyboards, and pushbuttons are all typical input devices for interactive computer kiosk.

The “personal computer” or simply “PC” is a term that is so often used it seems unlikely, at first, to require formal definition. However the precise scope of the term is sometimes vague. A PC is a computer whose size, and capabilities (and some have said price) make it useful for individuals, intended to be operated directly by an end user and capable of performing a variety of general purpose tasks, with no intervening computer operator. The PC may be a home computer, or may be found in an office, a medical facility or a research lab. The PC may often be connected to a local area network. The distinguishing characteristics of a PC are that the computer is primarily used, interactively, by one person at a time. This is opposite to the batch processing or time-sharing models which allowed large expensive systems to be used by many people, usually at the same time, or large data processing systems which required a full-time staff to operate. The PC can come in desktop models, notebook models, handheld models, and hybrids of these.

A “notebook computer”, or simply “notebook” within this application, is an extremely lightweight PC. Notebook computers typically weigh less than 6 pounds and are small enough to fit easily in a briefcase. Aside from size and portability, the principal difference between a notebook computer and a non-notebook personal computer (e.g. a desktop computer) is the display screen. Notebook computers use a variety of techniques, known as flat-pane technologies, to produce a lightweight and non-bulky display screen. Laptop computers and tablet PCs are two types of notebook computers. Usually all of the interface hardware needed to operate the notebook computer, such as parallel and serial ports, graphics card, sound channel, etc., are built in to a single unit. Most notebook computers contain batteries to facilitate operation without a readily available electrical outlet.

A “laptop computer”, or simply laptop, is, within this application, a subset of notebooks. A laptop will have a display and separate keyboard interface (e.g. “qwerty” keyboard), with the keyboard and the display typically hinged together. The term Laptop is sometimes used more broadly and equated with notebooks, but the term will have a narrower definition within this application.

A “Tablet PC” is a notebook, also called a panel computer, and was first introduced by Pen Computing in the early 90s with their PenGo Tablet Computer and popularized by Microsoft. The touch-screen or “graphics tablet/screen hybrid technology” technology of the tablet PHOTOCHROMIC allows the user to operate the computer with a stylus or digital pen, or a fingertip, instead of a keyboard or mouse. The tablet PC is particularly well suited to operate in Kiosk mode in light of the built in user interface provided with the tablet PC.

The input/output ports of a personal computer refer to the communications links through which the personal computers send and receive information, which generally include serial ports, parallel ports, wireless links or connectors (such as WI-FL and Bluetooth), and universal serial bus (USB) ports. In addition, some laptops have expansion slots for PCMCIA standard adaptor cards (Type I and Type II) that also form input/output ports.

In the clinical fields, physiologic parameters of subjects are typically viewed through a “medical monitor” that is defined as an automated medical device that senses a patient's vital signs through an associated sensor and displays the results. Medical monitors are typically highly specialized and suited solely for the designated monitoring tasks. The modern trend is to have multi-parameter medical monitors that can track and display different vital signs on a common display. This specialization has limited the applicability of many of these devices in the research applications. Further, this specialization has, in some applications, limited the portability of the medical monitor, limiting the applications to the hospital or clinic environment and resulting in the device being impractical for certain portable applications such as needed in the veterinary fields.

In the research field, physiologic parameters of subjects can, of course, be viewed through a “medical monitor” in the same manner as in current clinical fields. The assignee of this application has developed laptop and desktop PC based physiologic sensors that have been embraced by researchers. These devices, such as the MOUSE OX™ brand pulse oximeters, allow researchers to collect data on the physiologic parameters of the subjects such as mice on the researcher's laptop or desktop PC and to work with the data on their laptop or desktop PC. These laptop and desktop PC based physiologic sensor systems could, in theory, be operated in the clinical environments. However, the current laptop and desktop PC based physiologic sensor systems have not been widely adopted in the clinical fields because (1) there is a more significant space restriction associated with many clinical applications (i.e. there is no available desktop space and thus the system would require its own cart or stand), and (2) there is a need in clinical environments to avoid the program start up and selection procedures associated with general operating PCs as clinical technician are less inclined to work through an operating system to view and/or record the physiologic parameters.

A physiologic sensor within the meaning of this specification is a sensor that measures a parameter related to a physical characteristic of a living subject, such as a human or animal. The types of physiologic sensors include, for example, blood pressure sensors, blood flow sensors, blood glucose sensors, blood cholesterol sensors, heart sound sensors, EMG sensors, EEG sensors, EKG sensors, EOG sensors, pulse sensors, oxygenation sensors, pulse-oximetry sensors, blood perfusion sensors, respiration sensors (both pressure, flow and rate), temperature sensors, additional blood gas sensors (such as nitrogen partial pressure, carbon dioxide partial pressure, carbon monoxide partial pressure, oxygen partial pressure, and pH level), motion sensors, strain gauges, body position sensors, limb motion sensors and the like.

Respiratory sensors are a subset of physiologic sensors and refer to those sensors primarily measuring physical parameters of a subject indicative of respiration of the subject. A ventilation based sensor is, for example, a respiratory sensor. Cardiac sensors are a subset of physiologic sensors and refer to those sensors primarily measuring physical parameters of a subject indicative of cardiac function of the subject. A pulse oximeter is, for example, a cardiac sensor. However, as noted in detail below, a respiratory sensor can provide signals indicative of other physiologic parameters outside of respiration and a cardiac sensor can provide signals indicative of non-cardiac functions. For example the pulse oximeter can be used to calculate breathing rates.

U.S. Published Patent Application 2006-0264762 discloses a personal computer (PC) based physiologic monitor system that includes a personal computer having a display and an input/output port for attachment to an external device. The PC based system also includes a physiologic sensor coupled to the personal computer through the input/output port so that a modified output of the physiologic sensor is graphically displayed on the display. A controller, a portion of which is disposed in the personal computer, modifies the output of the physiologic sensor and provides a feedback control signal for modifying the output of the physiologic sensor. This disclosure is incorporated herein by reference.

There remains a need in the art to for a simple to simple to use physiologic sensor system effective for clinical and research applications.

### SUMMARY OF THE INVENTION

Some of the above objects are achieved with a portable modular kiosk based physiologic sensor system for clinical and research applications configured to simultaneously utilize multiple sensors with cross checking and cross calculation of physiologic parameters.

The term “cross calculating” within the meaning of this application references the calculation of a physiologic parameter in which the input from at least two sensors that are coupled to two distinct standard input ports on the PC are utilized in the calculation of the parameter.

The term “cross checking” within the meaning of this application references the calculation of a physiologic parameter in which the input from at least two sensors that are coupled to two distinct standard input ports on the PC are utilized independently in the separate calculation of the parameter and these two values are utilized to arrive at a given parameter value.

These and other advantages of the present invention will be clarified in the description of the preferred embodiments taken together with the attached drawings in which like reference numerals represent like elements throughout.

### BRIEF DESCRIPTION OF THE DRAWINGS

FIG. 1 is a schematic view of a portable modular kiosk based physiologic sensor system for clinical and research applications configured to simultaneously utilize multiple sensors with cross checking and cross calculation of physiologic parameters in accordance with the present invention;

FIG. 2 is a schematic section view of tablet computer sample display of the portable modular kiosk based physiologic sensor system according to one aspect of the present invention; and

FIG. 3 is a schematic view of a portable modular kiosk based physiologic sensor system for clinical and research applications configured to simultaneously utilize multiple sensors with cross checking and cross calculation of physiologic parameters in accordance with the present invention.

### DESCRIPTION OF THE PREFERRED EMBODIMENTS

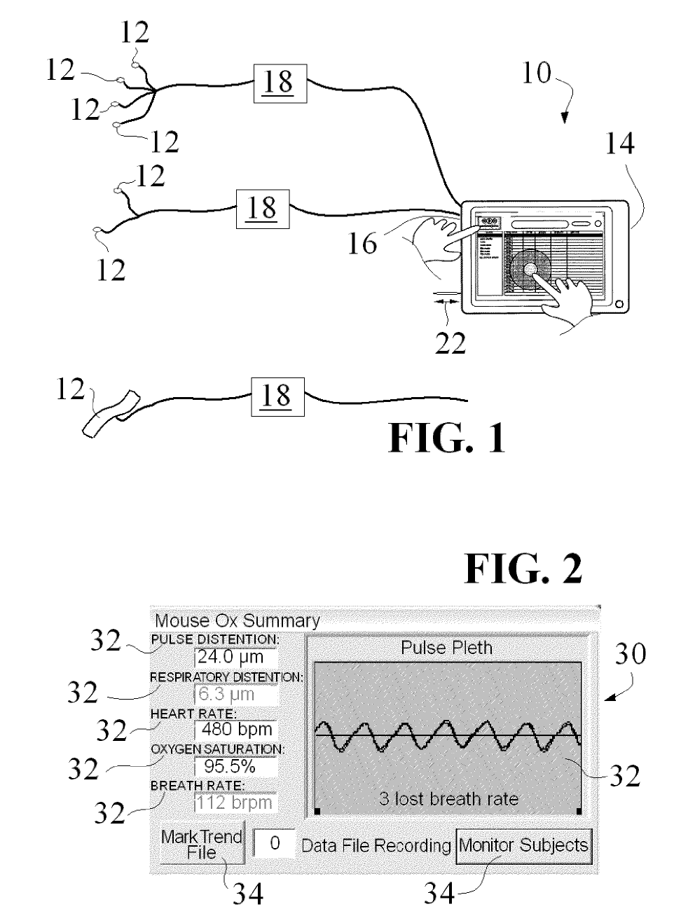

FIG. 1 is a schematic view of a portable modular kiosk based physiologic sensor system 10 for clinical and research applications configured to simultaneously utilize multiple sensors 12 with cross checking and cross calculation of physiologic parameters in accordance with the present invention.

One key component of the present invention is a PC computer 14 to which the multiple sensors 12 can be attached through conventional PC input ports 16. As noted above the input/output ports 16 of a personal computer 14 refer to the communications links through which the personal computers 14 send and receive information, which generally include serial ports, parallel ports, wireless links or connectors (such as WI-FL and Bluetooth), and universal serial bus (USB) ports 16. Where a physical connection is used (i.e. non-wireless), the USB port 16 will be the preferred connection for the present invention as several such input ports 16 are commonly provided on commercially available PC Computers 14.

The PC computer 14 of the present invention is preferably a notebook computer 14 such as a tablet PC 14 as shown. As noted above a notebook computer 14 is an extremely lightweight PC that typically weighs less than 6 pounds and is small enough to fit easily in a briefcase. Laptop computers and tablet PCs 14 are two types of notebook computers 14 for use in the present invention, wherein all of the interface hardware needed to operate the notebook computer 14, such as parallel and serial ports, graphics card, sound channel, etc., are built in to a single unit. The notebook computers 14 for implementing the present invention contain batteries to facilitate operation without a readily available electrical outlet.

In one embodiment of the present invention the system 10 according to the present invention is designed primarily for clinical applications. In the clinical application of the present invention the PC 14 is in the form of a Tablet PC 14, also called a panel computer, that incorporates a touch-screen 30 or “graphics tablet/screen hybrid technology” technology that allows the user to operate the computer with a stylus or digital pen 22, or a fingertip, instead of a keyboard or mouse. A joystick may be incorporated into the tablet 14 as a separate user input device.

In the clinical application of the present invention the Tablet PC 14 is operated in Kiosk mode. The Tablet PC 14 is particularly well suited to operate in Kiosk mode in light of the built in user interface provided with the tablet PC 14. One simple method of operating in Kiosk mode with the Tablet PC 14 is for the operating software to be launched at system start up. In a Microsoft Window® environment this is easily accomplished by dragging the operating software into the startup menu whereby it will be launched automatically at system startup. In this manner the system 10 is operable with simply a push of the on-off button.

One critical component of the present invention is a series of physiologic sensors 12 that are configured to be coupled to an input port 16 of the PC 14 such as through a USB port or through a wireless connection 18. Each sensor 12 is configured to measure a parameter related to a physical characteristic of a living subject, such as a human or animal, in particular a small mammal such as a mouse.

The types of physiologic sensors 12 include, for example, blood pressure sensors 12, blood flow sensors 12, blood glucose sensors 12, blood cholesterol sensors 12, heart sound sensors 12, EMG sensors 12, EEG sensors 12, EKG sensors 12, EOG sensors 12, pulse sensors 12, oxygenation sensors 12, pulse-oximetry sensors 12, blood perfusion sensors 12, respiration sensors (both pressure, flow and rate) 12, temperature sensors 12, additional blood gas sensors (such as nitrogen partial pressure, carbon dioxide partial pressure, carbon monoxide partial pressure, oxygen partial pressure, and pH level) 12, motion sensors 12, strain gauges 12, body position sensors 12, limb motion sensors 12 and the like.

For example, the MouseOx® sensor sold by the assignee of the present invention is one representative example of a sensor 12 for use in the system according to the present invention.

The sensors 12 will typically include an analog to digital converter (shown in coupling 18) between the sensor 12 elements that are coupled to the subject 20 and the PC coupling or port 16. Additionally the sensors 12 will occasionally need power. The sensors 12 may be plugged into a power supply through a separate power cord. However, in one aspect of the present invention separate power supply is incorporated into the sensor 12 to allow for elimination of the external power coupling and the need for a “close by” source of power, allowing the system to be particularly well suited for “field” operation. A separate method of eliminating the external power supply in the sensors 12 is to design the sensors 12 to draw power from the PC 14 through the coupling 18 to the PC 14. The elimination of the external power supply on the sensors 12 will assist in the portability of the system 10.

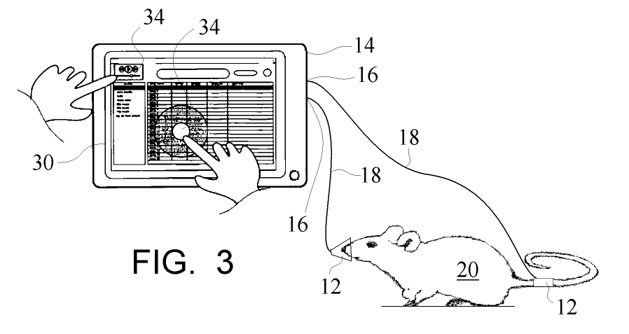

The modular aspect of the system 10 in accordance with the present invention is that a plurality of sensors 12 is provided for use in the system 10. It is anticipated that the user (clinician or researcher) will utilize a plurality of sensors 12 at the same time for any given subject 20 as shown in FIGS. 1 and 3. It is expected that these sensors 12 can be easily plugged into and removed from the PC 14. The PC 14 of the system 10 will recognize which sensors 12 are coupled to the input ports 16 and will configure the display 30 to display all of the relevant parameters at a designated display 32. The particular designated parameters display 32 that are calculated by the collection of sensors 12 (whether the parameters are calculated by individual sensors 12, cross checked with multiple sensors 12 or cross calculated from the output of two or more sensors 12). The display 30 can also include touch screen “buttons” or controls 34 for operation of the system 10.

Another aspect of the system 10 of the present invention is the cross checking of physiologic parameters determined by the system 10. In other words, for cross checking of a parameter, the calculation of the physiologic parameter uses the input from at least two sensors 12 that are coupled to two distinct standard input ports 16 on the PC 14 wherein these inputs are utilized independently in the separate calculation of the parameter and these two values are utilized to arrive at a given parameter value.

The concept of cross checking may be explained better by way of example. Breath rate of a subject 20 can be determined by both a ventilation based pressure sensor 12 and from a pulse oximeter 12. In this example the ventilation based sensor 12 is a respiratory sensor and the pulse oximeter is a cardiac sensor 12. Regardless the breath rate can be calculated by both sensors 12 independently and the Cross checking will use these two values in some fashion.

The use rules for cross checking may be that the dominant measurement rules. For example, the ventilation based sensor 12 can be considered a more accurate and robust measurement for this particular parameter, whereby the cross checking operates by having this be the dominant value. In other words if the ventilation based sensor 12 calculates the respiratory rate with a high degree of confidence then this is the value that is reported for this parameter and the pulse oximeter 12 reading or breath rate calculation is used if the respiratory sensor is not a confident reading. Further, the dominant reading can be compared to the less dominant reading and if there is a difference then the system can record that their may be an error in the non-dominant sensor. This can be important information since if the pulse oximeter is way off in the calculation of the breath rate then other readings may also be suspect. This cross checking system can thus be used as a sensor checking feature.

The use rules for cross checking may be an averaging of two calculated values to obtain a final parameter value, together with a checking of how far apart the two independent values are. The greater the variance in the independently obtained values the less confidence the system 10 should have in the accuracy of the final value. A threshold for the amount of difference that is acceptable can be utilized for each particular cross checked parameter.

The use rules for cross checking may include the use of threshold values and a discarding of those independently calculated parameter values that are outside the threshold. If both calculated parameters are outside the threshold then the system 10 can indicate that no value was calculated, or the system 10 may use the last known calculated parameter to display. If both calculated parameters are outside the threshold then the system 10 can further use one of the systems or methodologies described above such as averaging or dominant sensor rules.

The cross checking may be associated with more than two sensors 12, but the concept is not significantly different with three or more sensors 12 used to form a cross checked parameter.

Another aspect of the system of the present invention is the cross calculating of physiologic parameters determined by the system 10 wherein this defines the calculation of a physiologic parameter in which the input from at least two sensors 12 that are coupled to two distinct standard input ports 16 on the PC 14 are utilized in the calculation of the parameter. Cross calculation differs from cross checking in that the particular physiologic parameter cannot be calculated without the input from the two associated sensors.

An example of a cross calculated parameter is blood oxygenation/tidal volume. This parameter requires input from a pulse oximeter, for example, and a respiratory sensor. The present system contemplates one or more cross calculated parameters being made available to the user when two or more sensors are coupled to the PC. Pulse distention/tidal volume and breath distention/tidal volume would also represent cross calculated parameters available with the present invention that may be useful to researchers.

The cross calculating may be associated with more than two sensors 12, but the concept is not significantly different with three or more sensors 12 used to form a cross calculated parameter. Further the cross calculated parameters need not be simple ratios as presented in the example, but any combination of other parameters is contemplated.

In short the present invention provides a tool for clinicians, researchers, caregivers, educators and manufacturers that can be used in a number of distinct applications and although the present invention has been described with particularity herein, the scope of the present invention is not limited to the specific embodiment disclosed. It will be apparent to those of ordinary skill in the art that various modifications may be made to the present invention without departing from the spirit and scope thereof. The scope of the invention is not to be limited by the illustrative examples described above. The scope of the present invention is defined by the appended claims and equivalents thereto.

What is claimed is:

1. A portable modular kiosk based physiologic sensor system for clinical and research applications configured to simultaneously utilize multiple sensors with cross checking and cross calculation of physiologic parameters.

2. The kiosk based physiologic sensor system according to claim 1 wherein the system includes a notebook PC.

3. The kiosk based physiologic sensor system according to claim 2 wherein the notebook PC is a tablet PC.

4. The kiosk based physiologic sensor system according to claim 3 wherein the sensors include at least two sensors that are configured to be simultaneously coupled to the tablet PC through a USB port connection.

5. The kiosk based physiologic sensor system according to claim 4 wherein the system is configured for operation on small mammals.

6. The kiosk based physiologic sensor system according to claim 5 wherein the sensors include at least one pulse oximeter.

7. A portable modular physiologic sensor system comprising a PC which is configured to simultaneously utilize multiple sensors with cross checking of at least one physiologic parameter, wherein the cross checking includes the calculation of the physiologic parameter using the input from at least two sensors that are coupled to two distinct standard input ports on the PC, wherein these inputs are utilized independently in the separate calculation of the cross checked parameter and these two independently calculated values of the cross checked parameter are utilized to arrive at a given cross checked parameter value.

8. The modular physiologic sensor system according to claim 7 wherein the system includes a notebook PC.

9. The modular physiologic sensor system according to claim 8 wherein the notebook PC is a tablet PC.

10. The modular physiologic sensor system according to claim 9 wherein the sensors include at least two sensors that are configured to be simultaneously coupled to the tablet PC through a USB port connection.

11. The modular physiologic sensor system according to claim 10 wherein the system is configured for operation on small mammals.

12. The modular physiologic sensor system according to claim 11 wherein the sensors include at least one pulse oximeter.

13. A portable modular physiologic sensor system comprising a PC which is configured to simultaneously utilize multiple sensors with cross calculation of at least one physiologic parameter wherein the cross calculation of a physiologic parameter utilizes the input from at least two sensors that are coupled to two distinct standard input ports on the PC.

14. The modular physiologic sensor system according to claim 13 wherein the system includes a notebook PC.

15. The modular physiologic sensor system according to claim 14 wherein the notebook PC is a tablet PC.

16. The modular physiologic sensor system according to claim 15 wherein the sensors include at least two sensors that are configured to be simultaneously coupled to the tablet PC through a USB port connection.

17. The modular physiologic sensor system according to claim 16 wherein the system is configured for operation on small mammals.

18. The modular physiologic sensor system according to claim 17 wherein the sensors include at least one pulse oximeter.

19. The modular physiologic sensor system according to claim 13 wherein a PC which is configured to simultaneously utilize multiple sensors with cross checking of at least one physiologic parameter, wherein the cross checking includes the calculation of the physiologic parameter using the input from at least two sensors that are coupled to two distinct standard input ports on the PC, wherein these inputs are utilized independently in the separate calculation of the cross checked parameter and these two independently calculated values of the cross checked parameter are utilized to arrive at a given cross checked parameter value.

20. The modular physiologic sensor system according to claim 14 wherein the PC is a tablet PC.
